(19) United States
(12) Patent Application Publication
(10) Pub. No.: US 2024/0298971 A1
(43) Pub. Date: Sep. 12, 2024

(54) SENSOR SYSTEM AND SENSOR STRUCTURE

(71) Applicant: NIHON KOHDEN CORPORATION, Tokyo (JP)

(72) Inventors: Fumiyuki MATSUMURA, Tokorozawa-shi (JP); Minori HOSOI, Tokorozawa-shi (JP); Mariko KOBAYASHI, Tokorozawa-shi (JP); Hirohito IGARASHI, Tokorozawa-shi (JP); Hiroaki YAHAGI, Tokorozawa-shi (JP); Shota TANAKA, Tokorozawa-shi (JP)

(21) Appl. No.: 18/591,720

(22) Filed: Feb. 29, 2024

(30) Foreign Application Priority Data
Mar. 8, 2023 (JP) 2023-035354

(51) Int. Cl. A61B 5/00; A61B 5/11; G08B 21/04

(52) U.S. Cl. CPC A61B 5/6892; A61B 5/1113; A61B 5/1116; G08B 21/0461; A61B 2562/164; A61B 2562/227

(57) ABSTRACT

A sensor system includes a bedding sensor including a detector that detects vital signs information of a subject on bedding and an accommodation portion that accommodates the detector, and an information processing device configured to process the detected vital signs information and capable of being in an attached state in which the information processing device is attached to the bedding sensor and a separated state in which the information processing device is separated from the bedding sensor.

### CROSS-REFERENCE TO RELATED APPLICATIONS

The present application claims the benefit of priority of Japanese Patent Application No. 2023-035354, filed on Mar. 8, 2023, the content of which is incorporated herein by reference.

### TECHNICAL FIELD

The presently disclosed subject matter relates to a sensor system and a sensor structure.

### BACKGROUND ART

A sensor system disposed together with bedding such as a bed has been developed (see, for example, JP2019-213587A). The sensor system includes, for example, a sensor that detects a body motion of a subject on the bedding, and an information processing device that processes vital signs information detected by the sensor.

### SUMMARY OF INVENTION

It is desirable that such a sensor system can be disposed on various types of bedding.

The presently disclosed subject matter has been made in view of the above circumstance, and an object thereof is to provide a sensor system and a sensor structure that can be disposed on various types of bedding.

A sensor system according to an aspect of the presently disclosed subject matter includes
a bedding sensor including a detector that detects vital signs information of a subject on bedding and an accommodation portion that accommodates the detector, and
an information processing device configured to process the detected vital signs information and capable of being in an attached state in which the information processing device is attached to the bedding sensor and a separated state in which the information processing device is separated from the bedding sensor.

A sensor structure according to an aspect of the presently disclosed subject matter includes
a bedding sensor including a detector that detects vital signs information of a subject on bedding and an accommodation portion that accommodates the detector; and
an attachment tool configured to attach, to the bedding sensor, an information processing device that processes the detected vital signs information, wherein
the information processing device is capable of being in an attached state in which the information processing device is attached to the bedding sensor by the attachment tool and a separated state in which the information processing device is separated from the bedding sensor.

In the sensor system and the sensor structure according to the presently disclosed subject matter, since the information processing device can be in the attached state and the separated state, the attached state and the separated state can be changed according to a shape, a mechanism, a usage situation, or the like of the bedding. Therefore, the sensor system and the sensor structure can be disposed on various types of bedding.

### BRIEF DESCRIPTION OF DRAWINGS

FIG. 1 is a plan view illustrating an example of an overall configuration of a sensor system according to a first embodiment;

FIG. 2 is a diagram illustrating an example of a cross-section configuration of a part of the sensor system illustrated in FIG. 1 together with bedding;

FIG. 3 is a perspective view for illustrating a case when an information processing device illustrated in FIG. 1 is in an attached state;

FIG. 4 is a perspective view for illustrating a case when the information processing device illustrated in FIG. 3 is in a separated state;

FIG. 5 is a plan view illustrating another example of the sensor system when the information processing device illustrated in FIG. 1 is in the attached state;

FIG. 6 is a diagram illustrating a cross-section configuration of a main portion of the sensor system illustrated in FIG. 5;

FIG. 7A is a diagram illustrating an example of a configuration of a side surface of a plate-shaped member illustrated in FIG. 6 and the like;

FIG. 7B is a diagram illustrating an example of a configuration of the plate-shaped member together with a first surface of a bedding sensor illustrated in FIG. 5 and the like;

FIG. 8 is a block diagram illustrating an example of a configuration of the information processing device illustrated in FIG. 1 and the like;

FIG. 9 is a diagram illustrating an example of a cross-section configuration of a sensor system according to Modification 1;

FIG. 10 is a diagram illustrating an example of a cross-section configuration of a sensor system according to a second embodiment;

FIG. 11 is a plan view illustrating an example of a configuration of a second surface of the bedding sensor illustrated in FIG. 10;

FIG. 12 is a plan view illustrating another example of the configuration of the second surface of the bedding sensor illustrated in FIG. 11;

FIG. 13 is a diagram illustrating an example of a cross-section configuration of a sensor system according to Modification 2;

FIG. 14 is a diagram illustrating an example of a cross-section configuration of a sensor system according to Modification 3;

FIG. 15 is a diagram illustrating an example of a plane configuration of a sensor system according to Modification 4;

FIG. 16 is a diagram illustrating an example of a cross-section configuration of a sensor system according to a third embodiment;

FIG. 17 is a cross-section view illustrating another state of the sensor system illustrated in FIG. 16; and

FIG. 18 is a cross-section view illustrating another example of the information processing device in the attached state.

### DESCRIPTION OF EMBODIMENTS

Hereinafter, embodiments according to one aspect of the presently disclosed subject matter will be described. The presently disclosed subject matter is not limited to the following embodiments. In the drawings, the same elements are denoted by the same reference numerals, and the redundant description thereof is omitted. In addition, dimensional ratios in the drawings are exaggerated for convenience of the description, and may be different from actual ratios.

### First Embodiment

### Configuration of Sensor System 1

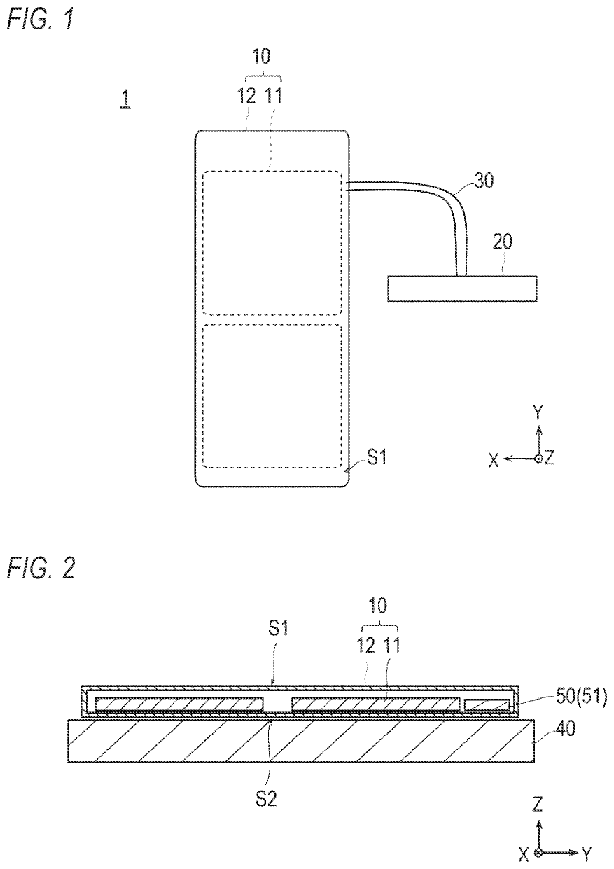

FIG. 1 and FIG. 2 illustrate an example of a configuration of a sensor system 1 according to a first embodiment of the presently disclosed subject matter. The sensor system 1 is disposed together with bedding 40 such as a bed. The sensor system 1 detects vital signs information from a subject on the bedding 40 over time. The subject sleeps on the bedding 40, for example. A user can monitor a state of the subject using the sensor system 1. The user is, for example, a medical worker, a caregiver, or a family member of the subject. The user may be the subject himself or herself.

The sensor system 1 includes, for example, a bedding sensor 10, an information processing device 20, a connection member 30, and a plate-shaped member 50. Here, the plate-shaped member 50 corresponds to a specific example of the attachment tool according to the presently disclosed subject matter, and a structure including at least the bedding sensor 10 and the plate-shaped member 50 corresponds to a specific example of a sensor structure according to the presently disclosed subject matter. In the following description, a long side direction, a short side direction, and a thickness direction of the substantially rectangular bedding 40 may be referred to as a Y direction, an X direction, and a Z direction, respectively. FIG. 1 illustrates an example of a plane (XY plane) configuration of the sensor system 1, and FIG. 2 illustrates a cross-section (YZ cross-section) configuration of a part of the sensor system 1 illustrated in FIG. 1 together with bedding 40.

The bedding sensor 10 includes a detector 11 and an accommodation portion 12 that accommodates the detector 11. The information processing device 20 acquires vital signs information of the subject detected by the detector 11 and processes the acquired vital signs information. The information processing device 20 is disposed so as to separate from the bedding sensor 10 and the bedding 40, for example (FIG. 1). The connection member 30 connects the detector 11 and the information processing device 20. The plate-shaped member 50 is used when the information processing device 20 is attached to the bedding sensor 10.

The bedding sensor 10 is used by being laid on or under the bedding 40. The bedding 40 is, for example, a bed, a futon, a mattress, a sheet, an overlay, or a cushion. The bedding sensor 10 may be disposed so as to be in contact with the subject, or the bedding 40 or the like may be disposed between the subject and the bedding sensor 10. The bedding sensor 10 laid on or under the bedding 40 has a first surface S1 and a second surface S2 that face the bedding 40 (FIG. 2). The first surface S1 and the second surface S2 are, for example, XY planes. For example, the bedding sensor 10 acquires vital signs information of the subject who lies on the first surface S1 side.

The detector 11 detects vital signs information of the subject on the bedding 40. The vital signs information detected by the detector 11 includes, for example, information related to a body motion of the subject. The detector 11 includes, for example, an air pad or a piezoelectric sensor. The bedding sensor 10 may include a plurality of detectors 11. The detector 11 may include both an air pad and a piezoelectric sensor.

The accommodation portion 12 accommodating the detector 11 covers, for example, an entire surface of the detector 11. The accommodation portion 12 has, for example, a bag shape. The first surface S1 and the second surface S2 includes, for example, a part of the accommodation portion 12. The accommodation portion 12 may cover a part of the detector 11. The accommodation portion 12 is made of, for example, cloth, vinyl, or plastic.

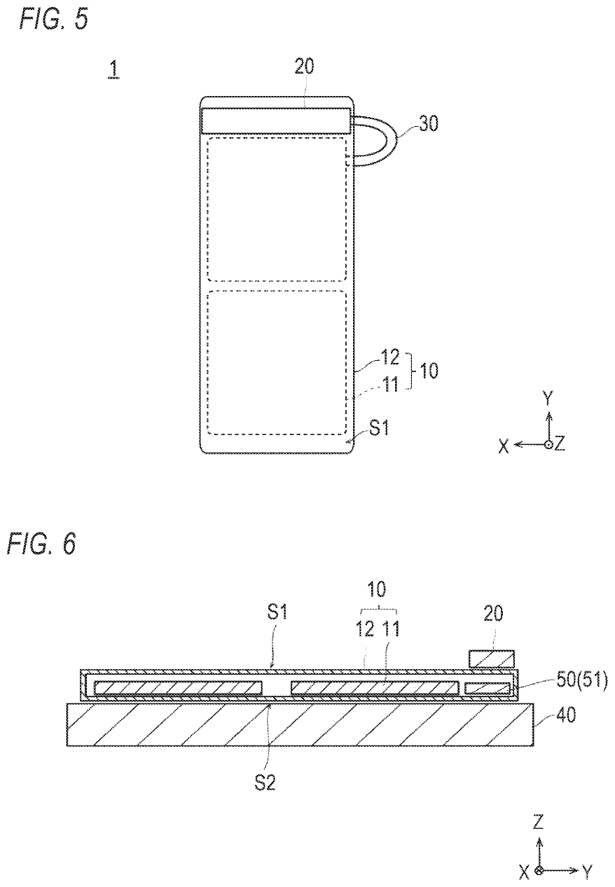

The vital signs information of the subject detected by the detector 11 is sent to the information processing device 20 via the connection member 30. The information processing device 20 has, for example, a box-shaped outer shape such as a rectangular parallelepiped or a cube. In the present embodiment, the information processing device 20 can be in either a separated state in which the information processing device 20 is disposed so as to separate from the bedding sensor 10 (FIG. 1 and FIG. 2) or an attached state in which the information processing device 20 is attached to the bedding sensor 10 (for example, FIG. 5 and FIG. 6 described later). Accordingly, as will be described in detail later, the user can change the attached state and the separated state according to a shape, a mechanism, a usage situation, or the like of the bedding, and the sensor system 1 can be disposed on various types of bedding.

Here, the information processing device 20 in the attached state refers to a state in which the information processing device 20 is integrated with the bedding sensor 10 and is held by the bedding sensor 10 directly or via an attachment tool described later. The information processing device 20 in the attached state is attached to, for example, the accommodation portion 12 of the bedding sensor 10.

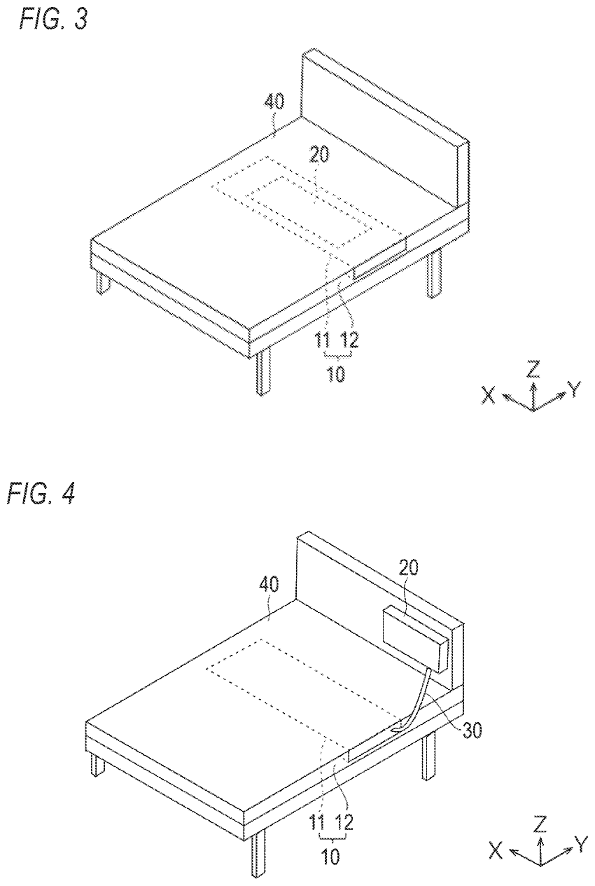

FIG. 3 illustrates an example of a configuration of the information processing device 20 in the attached state. The information processing device 20 in the attached state may be provided in the accommodation portion 12 together with the detector 11.

The information processing device 20 in the separated state refers to a state in which information processing device 20 is not held by the bedding sensor 10. At this time, for example, the accommodation portion 12 of the bedding sensor 10 and the information processing device 20 are disposed so as to separate from each other, and the accommodation portion 12 and the information processing device 20 can be moved separately. The information processing device 20 in the separated state is connected to the bedding sensor 10 (detector 11) only by the connection member 30, for example.

FIG. 4 illustrates an example of a configuration of the information processing device 20 in the separated state. The information processing device 20 in the separated state may be hung on, for example, a frame of the bedding 40 (bed).

FIG. 5 and FIG. 6 illustrate another example of the configuration of the information processing device 20 in the attached state. FIG. 5 illustrates a plane (XY plane) configuration of the sensor system 1 including the information processing device 20 in the attached state, and FIG. 6 illustrates a cross-section (YZ cross-section) configuration of a main portion of the sensor system 1 illustrated in FIG. 5. The information processing device 20 in the attached state may be attached to, for example, the first surface S1 of the bedding sensor 10 (FIG. 6). The information processing device 20 is attached to the bedding sensor 10 by, for example, the plate-shaped member 50 provided in the bedding sensor 10. Here, the plate-shaped member 50 corresponds to a specific example of the attachment tool according to the presently disclosed subject matter.

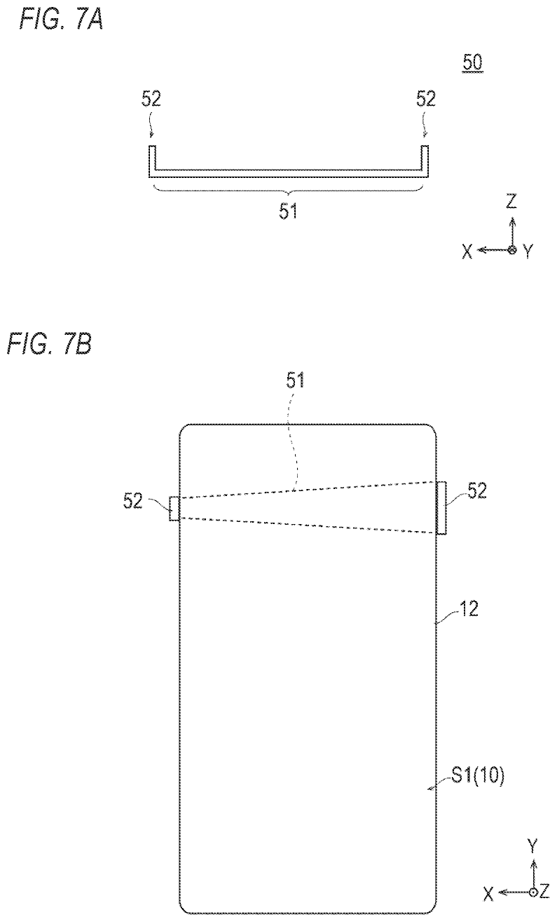

FIG. 7A and FIG. 7B illustrate an example of a configuration of the plate-shaped member 50. FIG. 7A illustrates an example of a configuration of a side surface (XZ plane) of the plate-shaped member 50, and FIG. 7B illustrates an example of the configuration of the plate-shaped member 50 together with the first surface S1 of the bedding sensor 10. The plate-shaped member 50 includes, for example, a flat portion 51 and a pair of claw portions 52. The plate-shaped member 50 attaches the information processing device 20 to the bedding sensor 10 so as to enable a change to the separated state. In other words, the user can freely attach and detach the information processing device 20 to and from the bedding sensor 10 using the plate-shaped member 50.

The flat portion 51 has, for example, a quadrangular plane (XY plane) shape such as a trapezoid. The flat portion 51 is inserted, for example, between the first surface S1 and the second surface S2 of the bedding sensor 10 along the X direction. For example, the flat portion 51 is accommodated in the accommodation portion 12 together with the detector 11. The flat portion 51 is fixed to a predetermined position of the accommodation portion 12. In the attached state, the information processing device 20 is placed on the flat portion 51 via the accommodation portion 12.

The pair of claw portions 52 are provided at both ends of the flat portion 51 in the X direction, and are erected in the Z direction (FIG. 7A). The pair of claw portions 52 have, for example, a predetermined size in the Y direction. The pair of claw portions 52 are disposed to be exposed from the accommodation portion 12, for example (FIG. 7B). The pair of claw portions 52 are configured to allow the information processing device 20 to be attached and detached, and for example, a distance (distance in the X direction) between the pair of claw portions 52 is substantially the same as a size of the information processing device 20 in the X direction. In the attached state, the information processing device 20 is fitted between the pair of claw portions 52, and the information processing device 20 is held by the bedding sensor 10.

The information processing device 20 may include a recess or the like having a shape corresponding to the pair of claw portions 52. For example, the recess or the like is fixed to the claw portion 52. Here, the recess or the like corresponds to a specific example of a fixing portion according to the presently disclosed subject matter.

For example, by the user inserting the information processing device 20 between the pair of claw portions 52, the information processing device 20 is fitted between the pair of claw portions 52 and is brought into the attached state. By the user pulling out the information processing device 20 in the attached state from between the pair of claw portions 52, the information processing device 20 is disengaged from between the pair of claw portions 52 and is brought into the separated state.

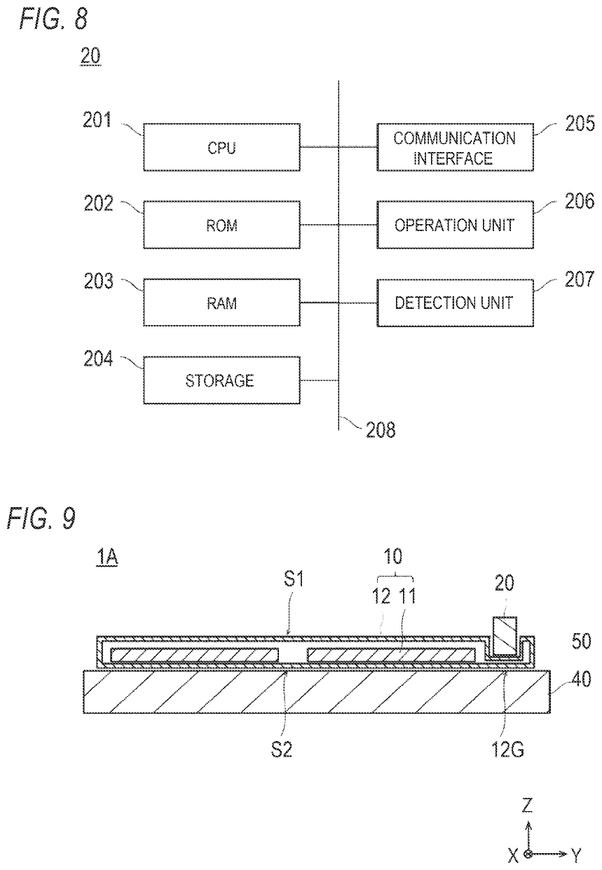

FIG. 8 is a block diagram illustrating a schematic configuration of the information processing device 20. The information processing device 20 includes a central processing unit (CPU) 201, a read only memory (ROM) 202, a random access memory (RAM) 203, a storage 204, a communication interface 205, an operation unit 206, and a detection unit 207. These components are communicably connected to each other via a bus 208.

The CPU 201 controls the bedding sensor 10 and performs various types of arithmetic processing according to a program recorded in the ROM 202 or the storage 204. For example, the CPU 201 causes the bedding sensor 10 to detect the vital signs information of the subject on the bedding 40, and causes the information processing device 20 to process the detected vital signs information and calculate a predetermined parameter representing the state of the subject. For example, the information processing device 20 calculates a pulse, a heart rate waveform, a heart rate, a pulse wave, a respiration rate, a respiration waveform, a heart sound, a bed-leaving state, a snoring state, a sleep-talking state, a body motion waveform, a psychological stress index, an index indicating a sleep state, or the like of the subject based on the vital signs information of the subject detected by the bedding sensor 10. For example, the CPU 201 extracts a signal in a predetermined frequency band by filtering a signal acquired from the detection unit 207, and calculates a predetermined parameter based on the extracted signal.

The ROM 202 stores various programs and various data for calculating predetermined parameters. The RAM 203 temporarily stores programs and data as a work area.

The storage 204 stores various programs including an operating system and various data. For example, the storage 204 is installed with an application for transmitting and receiving various kinds of information to and from the bedding sensor 10 and for determining a predetermined parameter calculated based on the vital signs information acquired from the bedding sensor 10.

The communication interface 205 is an interface for communicating with other devices. As the communication interface 205, a communication interface based on various wired or wireless standards is used. For example, the communication interface 205 is connected to the connection member 30. The predetermined parameter calculated by the information processing device 20 may be transmitted to another terminal device via the communication interface 205.

The operation unit 206 includes, for example, an operation button, and receives various inputs from the user. For example, when the user inputs an instruction to the operation unit 206, detection of vital signs information by the bedding sensor 10 is started. The operation unit 206 may be implemented by a touch panel display or the like. The information processing device 20 may include a display unit, separate from the operation unit 206, for displaying information related to the calculated predetermined parameter.

The detection unit 207 detects vital signs information of the subject sent from the bedding sensor 10 via the connection member 30, and generates an electric signal based on the vital signs information. For example, the CPU 201 calculates a predetermined parameter using the electric signal generated by the detection unit 207. For example, the detection unit 207 may include a piezoelectric element. The piezoelectric element detects, for example, a pressure of air sent from the bedding sensor 10 via the connection member 30.

The connection member 30 that connects the information processing device 20 and the detector 11 plays a role of transmitting the vital signs information of the subject detected by the detector 11 to the information processing device 20. The connection member 30 includes, for example, a wiring that electrically connects the detector 11 and the information processing device 20, or a pipe that allows a fluid such as air to flow from the detector 11 to the information processing device 20.

### Effects of Sensor System 1

In the sensor system 1 according to the present embodiment, the information processing device 20 can be in either the attached state in which the information processing device 20 is attached to the bedding sensor 10 or the separated state in which the information processing device 20 is separated from the bedding sensor 10. Specifically, by fitting the information processing device 20 between the pair of claw portions 52, the information processing device 20 is held by the bedding sensor 10 and is brought into the attached state. By pulling out the information processing device 20 in the attached state from between the pair of claw portions 52, the information processing device 20 in the attached state is not held by the bedding sensor 10 and is brought into the separated state. In this way, since the information processing device 20 can be in either the attached state or the separated state, the information processing device 20 can be changed in the attached state and the separated state according to a shape, a mechanism, a usage situation, or the like of the bedding 40, and can be disposed on various types of bedding. Hereinafter, the effects will be described.

The shape, mechanism, and the like of bedding used in each medical institution and the like are different. For example, different medical institutions have different beds with or without reclining mechanisms, with or without moving mechanisms, with or without folding mechanisms, and with or without removable fences. Further, different medical institutions have different widths, thicknesses, and hardness of mattresses or the like. For example, when the information processing device remains in either the attached state or the separated state and cannot be changed from one to the other, installing the sensor system on the bedding may be difficult. For example, in a case in which the information processing device remains in the attached state, when the fence of the bed is removed, the information processing device may be caught in the fence and damaged.

In contrast, in the sensor system 1, since the information processing device 20 can be in either the attached state or the separated state, the user can easily change the attached state and the separated state according to the shape, the mechanism, the usage situation, or the like of the bedding 40. For example, when the fence of the bed is removed, the information processing device 20 may be once set in the separated state, and after the fence is removed, the information processing device 20 may be set in the attached state. In this way, the sensor system 1 can be disposed on various types of bedding 40 regardless of whether the fence can be removed.

Hereinafter, modifications of the sensor system 1 described in the first embodiment and other embodiments will be described. In the following, in order to avoid duplication of description, detailed description of the same configuration as each configuration of the sensor system 1 described in the first embodiment will be omitted.

### Modification 1

FIG. 9 illustrates an example of a cross-section (YZ cross-section) configuration of a sensor system 1A according to Modification 1 together with the bedding 40. In the sensor system 1A, the information processing device 20 is attached to the bedding sensor 10 by a groove portion 12G provided in the bedding sensor 10. That is, here, the groove portion 12G corresponds to a specific example of the attachment tool according to the presently disclosed subject matter. Except for this point, the sensor system 1A has the same configuration as the sensor system 1 described in the first embodiment, and achieves the same effects.

The groove portion 12G is provided, for example, on the first surface S1 of the bedding sensor 10 along the X direction. The groove portion 12G is provided, for example, in the accommodation portion 12. The groove portion 12G is configured to allow the information processing device 20 to be fitted therein. In the attached state, the information processing device 20 is fitted into the groove portion 12G, and the information processing device 20 is held by the bedding sensor 10.

The information processing device 20 may include a convex portion or the like having a shape corresponding to the groove portion 12G. For example, the convex portion or the like is fixed to the groove portion 12G. Here, the convex portion or the like corresponds to a specific example of the fixing portion according to the presently disclosed subject matter.

For example, by the user inserting the information processing device 20 into the groove portion 12G while sliding the information processing device 20 in the X direction, the information processing device 20 is fitted into the groove portion 12G and is brought into the attached state. By the user sliding the information processing device 20 in the attached state in the X direction and pulling out the information processing device 20 from the groove portion 12G, the information processing device 20 is brought into the separated state.

Also in the sensor system 1A according to Modification 1, as described in the first embodiment, since the information processing device 20 can be in either the attached state or the separated state, the user can easily change the attached state and the separated state according to the shape, the mechanism, the usage situation, or the like of the bedding 40.

Therefore, the sensor system 1A can be disposed on various types of bedding 40.

### Second Embodiment

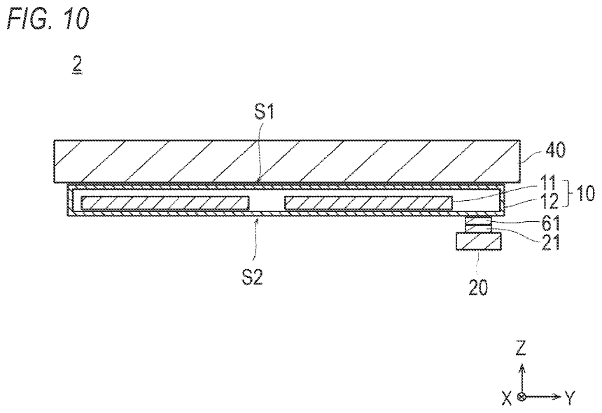

FIG. 10 illustrates an example of a cross-section (YZ cross-section) configuration of a sensor system 2 according to a second embodiment together with the bedding 40. In the sensor system 2, the information processing device 20 is attached to the second surface S2 of the bedding sensor 10. Except for this point, the sensor system 2 has the same configuration as the sensor system 1 described in the first embodiment, and achieves the same effects.

The information processing device 20 in the attached state is attached to the second surface S2 of the bedding sensor 10 by a first adhesive member 61, for example. That is, here, the first adhesive member 61 corresponds to a specific example of the attachment tool according to the presently disclosed subject matter. The first adhesive member 61 is configured to adhere the bedding sensor 10 and the information processing device 20.

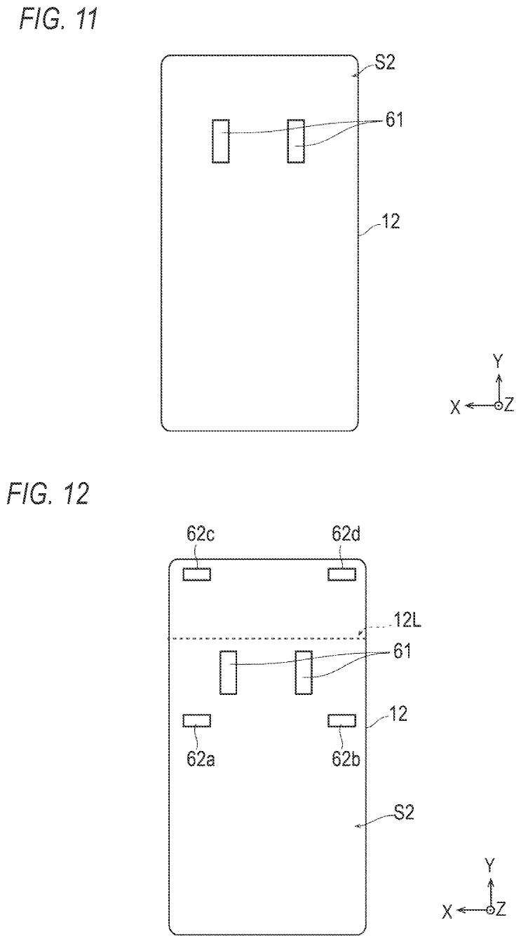

FIG. 11 illustrates an example of the configuration of the first adhesive member 61 provided on the second surface S2 of the bedding sensor 10. For example, a plurality of first adhesive members 61 are provided on the second surface S2. The first adhesive member 61 includes, for example, a hook-and-loop fastener or a magnet. At this time, the information processing device 20 includes a fixing portion 21 fixed to the first adhesive member 61. The fixing portion 21 includes, for example, a hook-and-loop fastener or a magnet. In the attached state, for example, by adhering the fixing portion 21 to each of the plurality of first adhesive members 61, the information processing device 20 is held by the bedding sensor 10.

FIG. 12 illustrates another example of the configuration of the second surface S2 of the bedding sensor 10. The second surface S2 of the bedding sensor 10 may be provided with second adhesive members 62a, 62b, 62c, and 62d together with the first adhesive member 61. For example, the second adhesive member 62a is adhered to the second adhesive member 62c, and the second adhesive member 62b is adhered to the second adhesive member 62d. Accordingly, the second surface S2 of the bedding sensor 10 bends along a line 12L, and the information processing device 20 in the attached state is covered by the second surface S2.

In the sensor system 2, for example, by the user adhering the fixing portion 21 of the information processing device 20 to the first adhesive member 61, the information processing device 20 is brought into the attached state. By the user peeling off the fixing portion 21 of the information processing device 20 in the attached state from the first adhesive member 61, the information processing device 20 is brought into the separated state.

Also in the sensor system 2 according to the second embodiment, as described in the first embodiment, since the information processing device 20 can be in either the attached state or the separated state, the user can easily change the attached state and the separated state according to the shape, the mechanism, the usage situation, or the like of the bedding 40.

Therefore, the sensor system 2 can be disposed on various types of bedding 40.

### Modification 2

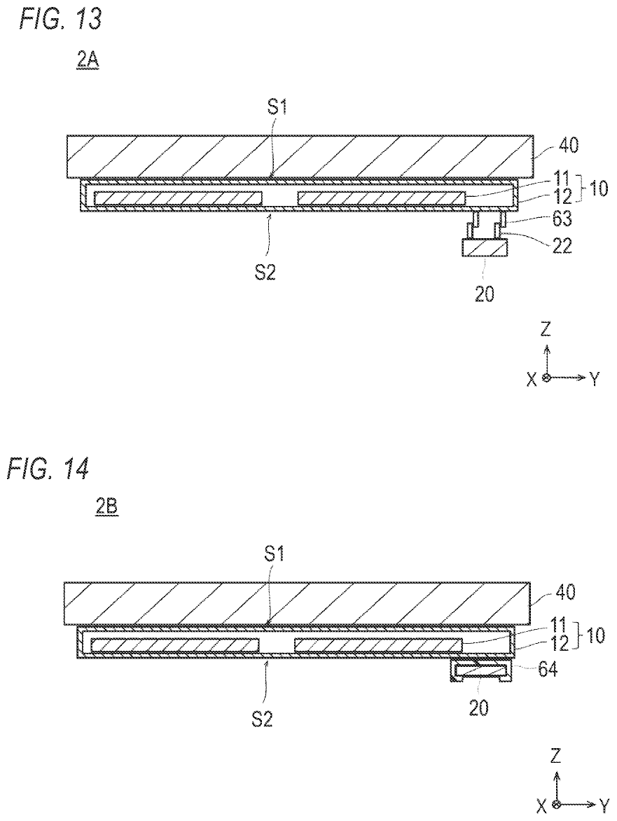

FIG. 13 illustrates an example of a cross-section (YZ cross-section) configuration of a sensor system 2A according to Modification 2 together with the bedding 40. In the sensor system 2A, the second surface S2 of the bedding sensor 10 is provided with a protrusion member 63. The information processing device 20 is attached to the bedding sensor 10 by the protrusion members 63. That is, here, the protrusion member 63 corresponds to a specific example of the attachment tool according to the presently disclosed subject matter. Except for this point, the sensor system 2A has the same configuration as the sensor system 2 described in the second embodiment, and achieves the same effects.

The protrusion member 63 is provided so as to protrude from the second surface S2. The protrusion member 63 is fixed to the second surface S2 by, for example, an adhesive (not illustrated). The second surface S2 may be provided with a plurality of protrusion members 63.

The protrusion member 63 is configured to hold the information processing device 20. At this time, the information processing device 20 includes, for example, a fixing portion 22 fixed to the protrusion member 63. The protrusion member 63 and the fixing portion 22 have, for example, hook shapes that can be engaged with each other. The protrusion member 63 is, for example, a hook or a clasp. In the attached state, by engaging the fixing portion 22 of the information processing device 20 with the protrusion member 63, the information processing device 20 is held by the bedding sensor 10.

For example, by the user engaging the fixing portion 21 of the information processing device 20 with the protrusion member 63, the information processing device 20 is brought into the attached state. By the user removing the fixing portion 21 of the information processing device 20 in the attached state from the protrusion member 63, the information processing device 20 is brought into the separated state.

Also in the sensor system 2A according to Modification 2, as described in the second embodiment, since the information processing device 20 can be in either the attached state or the separated state, the user can easily change the attached state and the separated state according to the shape, the mechanism, the usage situation, or the like of the bedding 40. Therefore, the sensor system 2A can be disposed on various types of bedding 40.

### Modification 3

FIG. 14 illustrates an example of a cross-section (YZ cross-section) configuration of a sensor system 2B according to Modification 3 together with the bedding 40. In the sensor system 2B, the second surface S2 of the bedding sensor 10 is provided with a casing 64. The information processing device 20 is attached to the bedding sensor 10 by the casing 64. That is, here, the casing 64 corresponds to a specific example of the attachment tool according to the presently disclosed subject matter. Except for this point, the sensor system 2B has the same configuration as the sensor system 2 described in the second embodiment, and achieves the same effects.

The casing 64 is configured to accommodate the information processing device 20. For example, the casing 64 covers a part of the information processing device 20 and accommodates the information processing device 20. For example, the casing 64 covers the information processing device 20 from one main surface to an outer peripheral portion of the other main surface, and exposes a central portion of the other main surface. The casing 64 may cover an entire surface of the information processing device 20. The casing 64 is made of, for example, a flexible material such as a resin material. The casing 64 is fixed to the second surface S2 by, for example, an adhesive (not illustrated). In the attached state, for example, by accommodating the information processing device 20 in the casing 64, the information processing device 20 is held by the bedding sensor 10.

For example, by the user accommodating the information processing device 20 in the casing 64, the information processing device 20 is brought into the attached state. By the user taking out the information processing device 20 in the attached state from the casing 64, the information processing device 20 is brought into the separated state.

Also in the sensor system 2B according to Modification 3, as described in the first embodiment, since the information processing device 20 can be in either the attached state or the separated state, the user can easily change the attached state and the separated state according to the shape, the mechanism, the usage situation, or the like of the bedding 40. Therefore, the sensor system 2B can be disposed on various types of bedding 40.

### Modification 4

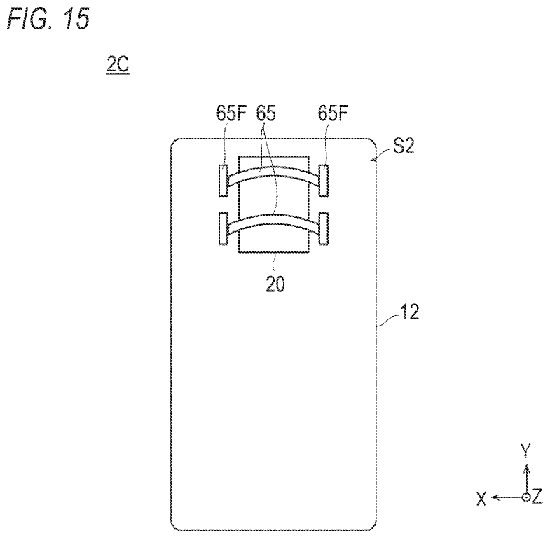

FIG. 15 illustrates an example of a plane (XY plane) configuration of a sensor system 2C according to Modification 4. In the sensor system 2C, a support member 65 is provided on the second surface S2 of the bedding sensor 10. The information processing device 20 is attached to the bedding sensor 10 by the support member 65. That is, here, the support member 65 corresponds to a specific example of the attachment tool according to the presently disclosed subject matter. Except for this point, the sensor system 2C has the same configuration as the sensor system 2 described in the second embodiment, and achieves the same effects.

The support member 65 is provided on the second surface S2 of the bedding sensor 10, and is configured to support the information processing device 20. The support member 65 includes, for example, a plurality of belt-shaped members, and both ends of each of the plurality of belt-shaped members are fixed to the second surface S2 of the bedding sensor 10 by fixtures 65F. The plurality of belt-shaped members are made of, for example, an elastic material. For example, a rubber band or the like can be used as the support member 65. In the attached state, for example, by supporting the information processing device 20 by the support member 65, the information processing device 20 is held by the bedding sensor 10.

For example, by the user placing the information processing device 20 between the support member 65 and the second surface S2 of the bedding sensor 10 and causing the support member 65 to support the information processing device 20, the information processing device 20 is brought into the attached state. By the user taking out the information processing device 20 in the attached state from between the support member 65 and the second surface S2 of the bedding sensor 10, the information processing device 20 is brought into the separated state.

Also in the sensor system 2C according to Modification 4, as described in the first embodiment, since the information processing device 20 can be in either the attached state or the separated state, the user can easily change the attached state and the separated state according to the shape, the mechanism, the usage situation, or the like of the bedding 40. Therefore, the sensor system 2C can be disposed on various types of bedding 40.

### Third Embodiment

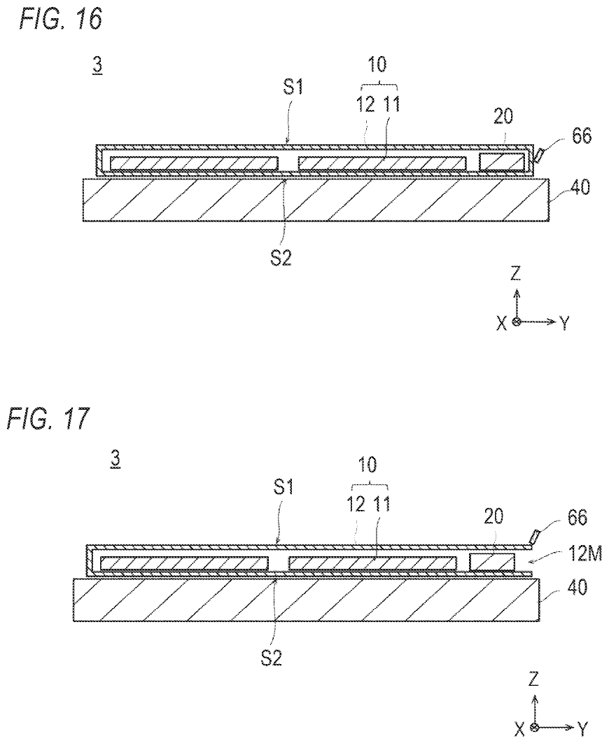

FIG. 16 illustrates an example of a cross-section (YZ cross-section) configuration of a sensor system 3 according to a third embodiment together with the bedding 40. In the sensor system 3, the information processing device 20 in the attached state is attached between the first surface S1 and the second surface S2 of the bedding sensor 10. Except for this point, the sensor system 3 has the same configuration as the sensor system 1 described in the first embodiment, and achieves the same effects.

FIG. 17 illustrates another state of the sensor system 3 illustrated in FIG. 16. For example, the accommodation portion 12 of the sensor system 1 is provided with an opening 12M capable of receiving the information processing device 20. The sensor system 1 includes an opening/closing member 66 configured to open/close the opening 12M, and the information processing device 20 in the attached state is attached between the first surface S1 and the second surface S2 of the bedding sensor 10 by the opening/closing member 66 (FIG. 16). The opening/closing member 66 includes, for example, a chuck, a fastener, a zipper, a button, a buckle, or a belt. Here, the opening/closing member 66 corresponds to a specific example of the attachment tool according to the presently disclosed subject matter.

In the sensor system 3, for example, by the user inserting the information processing device 20 between the first surface S1 and the second surface S2 of the bedding sensor 10 from the opening 12M, and closing the opening 12M using the opening/closing member 66, the information processing device 20 is brought into the attached state. By the user taking out the information processing device 20 in the attached state from the opening 12M opened using the opening/closing member 66, the information processing device 20 is brought into the separated state.

Also in the sensor system 3 according to the third embodiment, as described in the description in the first embodiment, since the information processing device 20 can be in either the attached state or the separated state, the user can easily change the attached state and the separated state according to the shape, the mechanism, the usage situation, or the like of the bedding 40. Therefore, the sensor system 3 can be disposed on various types of bedding 40.

The sensor system and the sensor structure according to the presently disclosed subject matter have been described above with reference to the embodiments and the modifications. However, the presently disclosed subject matter can be appropriately added, modified, or omitted by those skilled in the art within the scope of the technical idea thereof. For example, the configurations, shapes, sizes, and the like of the respective units of the sensor system and the sensor structure described in the above embodiments and the like are merely examples, and other configurations, shapes, sizes, and the like may be used.

For example, in the above embodiments and the like, specific examples of the attachment tool have been described. However, the attachment tool may be any attachment tool that can attach the information processing device 20 to the bedding sensor 10 so as to be able to be in the attached state and the separated state.

Further, in the above embodiments and the like, an example in which the information processing device 20 in the attached state is attached to the first surface S1, the second surface S2, or between the first surface S1 and the second surface S2 of the bedding sensor 10 has been described, and the information processing device 20 in the attached state may be attached to other portions of the bedding sensor 10.

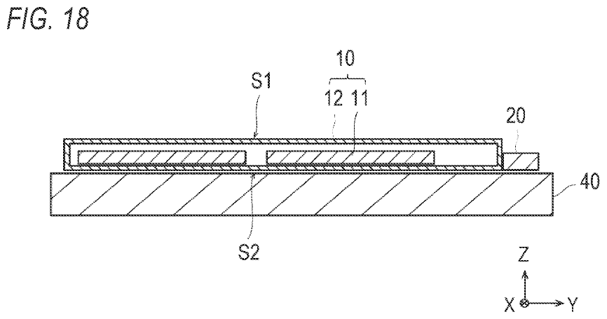

FIG. 18 illustrates an example of the information processing device 20 in the attached state. In this way, the information processing device 20 in the attached state may be attached to a surface that connects the first surface S1 and the second surface S2 of the bedding sensor 10.

Further, in the above embodiments and the like, an example in which the bedding sensor 10 has a substantially rectangular planar shape has been described. However, the bedding sensor 10 may have other planar shapes such as a square shape or an elliptical shape.

Further, in the above embodiments and the like, an example has been described in which a subject lies on the first surface S1 side of the bedding sensor 10 and the bedding sensor 10 acquires vital signs information of the subject. However, the bedding sensor 10 may acquire vital signs information of the subject who lies on the second surface S2 side.

Further, in the above embodiments and the like, an example in which the sensor system 1 and the like include the connection member 30 has been described. However, the detector 11 and the information processing device 20 may be wirelessly connected, and the sensor system 1 and the like may not include the connection member 30. At this time, the information processing device 20 in the separated state may not be connected to the bedding sensor 10 by the connection member 30.

Further, in the above embodiments and the like, an example in which the information processing device 20 in the attached state is attached to the accommodation portion 12 has been described. However, the information processing device 20 in the attached state may be attached to the detector 11 of the bedding sensor 10.

What is claimed is:

1. A sensor system comprising:
a bedding sensor including a detector that detects vital signs information of a subject on bedding and an accommodation portion that accommodates the detector; and
an information processing device configured to process the detected vital signs information and capable of being in an attached state in which the information processing device is attached to the bedding sensor and a separated state in which the information processing device is separated from the bedding sensor.

2. The sensor system according to claim 1, wherein
the information processing device in the attached state is attached to the bedding sensor by an attachment tool.

3. The sensor system according to claim 2, wherein
the information processing device in the attached state includes a fixing portion fixed to the attachment tool.

4. The sensor system according to claim 2, wherein
the bedding sensor is laid on or under the bedding and has a first surface and a second surface that face the bedding.

5. The sensor system according to claim 4, wherein
the information processing device in the attached state is attached to the first surface or the second surface of the bedding sensor by the attachment tool.

6. The sensor system according to claim 5, wherein
the attachment tool includes a protrusion member that is arranged at the bedding sensor so as to protrude from the first surface or the second surface and is configured to hold the information processing device.

7. The sensor system according to claim 5, wherein
the information processing device in the attached state is attached between the first surface and the second surface of the bedding sensor by the attachment tool.

8. The sensor system according to claim 2, wherein
the attachment tool includes a plate-shaped member arranged at the bedding sensor, and
the plate-shaped member is provided with a pair of claw portions configured to allow the information processing device to be attached and detached.

9. The sensor system according to claim 2, wherein
the attachment tool includes an adhesive member that is arranged at the bedding sensor and is configured to adhere the information processing device.

10. The sensor system according to claim 2, wherein
the attachment tool includes a casing that is arranged at the bedding sensor and is configured to accommodate the information processing device.

11. The sensor system according to claim 2, wherein
the attachment tool includes a groove portion that is provided in the bedding sensor and is configured to allow the information processing device to be fitted therein.

12. The sensor system according to claim 2, wherein
the accommodation portion is provided with an opening capable of receiving the information processing device, and
the attachment tool includes an opening/closing member configured to open/close the opening.

13. The sensor system according to claim 2, wherein
the attachment tool includes a support member that is arranged at the bedding sensor and is configured to support the information processing device.

14. The sensor system according to claim 1, wherein
the information processing device in the attached state is attached to the accommodation portion.

15. The sensor system according to claim 1, further comprising:
a connection member configured to connect the detector and the information processing device.

16. The sensor system according to claim 1, wherein
the vital signs information includes information related to a body motion of the subject.

17. A sensor structure comprising:
a bedding sensor including a detector that detects vital signs information of a subject on bedding and an accommodation portion that accommodates the detector; and
an attachment tool configured to attach, to the bedding sensor, an information processing device that processes the detected vital signs information, wherein
the information processing device is capable of being in an attached state in which the information processing device is attached to the bedding sensor by the attachment tool and a separated state in which the information processing device is separated from the bedding sensor.
